(19) United States
(12) Patent Application Publication
(10) Pub. No.: US 2025/0228705 A1
(43) Pub. Date: Jul. 17, 2025

(54) ROBOTIC CAPSULOTOMY

(71) Applicant: FORSIGHT ROBOTICS LTD., Yokneam Illit (IL)

(72) Inventor: Daniel Glozman, Kfar Yona (IL)

(21) Appl. No.: 19/097,061

(22) Filed: Apr. 1, 2025

Related U.S. Application Data
(63) Continuation of application No. PCT/IB2023/059694, filed on Sep. 28, 2023.
(60) Provisional application No. 63/412,475, filed on Oct. 2, 2022.

(51) Int. Cl. A61F 9/007; A61B 17/00; A61B 18/00; A61B 18/08; A61B 34/20; A61B 34/30

(52) U.S. Cl. CPC A61F 9/00754; A61B 34/30; A61B 2017/00694; A61B 2018/00601; A61B 18/082; A61B 2034/2055; A61B 2034/2065; A61B 2034/302

(57) ABSTRACT

Apparatus and methods are described for performing a capsulotomy procedure on a patient's eye. A diathermic capsulotomy tool includes a diathermic cutting element at its tip. An imaging system images the diathermic capsulotomy tool and the patient's eye. A computer processor drives a robotic unit to insert the diathermic capsulotomy tool into the patient's eye via an incision in a cornea of the patient's eye, such that the tip of the tool is disposed within the patient's eye and a remote center of motion location of the tool is disposed within the incision. The computer processor drives the robotic unit to move the cutting element in a circular motion while maintaining the remote center of motion location of the tool within the incision. Other applications are also described.

### CROSS-REFERENCES TO RELATED APPLICATIONS

The present application is a continuation of PCT Application PCT/IB2023/059694 to Glozman, filed Sep. 28, 2023, entitled “Robotic Capsulotomy,” which claims priority from U.S. Provisional Patent Application No. 63/412,475 to Glozman, filed Oct. 2, 2022, entitled “Robotic Capsulotomy,” which is incorporated herein by reference.

### FIELD OF EMBODIMENTS OF THE INVENTION

Some applications of the present invention generally relate to medical apparatus and methods. Specifically, some applications of the present invention relate to apparatus and methods for performing microsurgical procedures in a robotic manner.

### BACKGROUND

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and their face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye, in order to further anesthetize the eye. Following this step, a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulotomy or capsulorhexis (in the present application, these terms are used interchangeably), a part of the anterior lens capsule is removed. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening). Most commonly, capsulorhexis is performed using a technique called continuous curvilinear capsulorhexis, in which a bent needle is used to cut a tear in the anterior lens capsule, and the tear is then extended around the anterior lens capsule using the same needle and/or forceps. It is typically important that the anterior lens capsule is cut precisely and that the cut is centered in order to maintain the intraocular lens in a centralized position. There is evidence that even slight decentralization of the intraocular lens can decrease visual acuity and can lead to astigmatism.

Subsequently, it is common for a fluid wave to be injected via the corneal incision, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. Further typically, a separate tool is used to perform suction during the phacoemulsification. When the phacoemulsification is complete, the remaining lens cortex (i.e., the outer layer of the lens) material is aspirated from the capsule. During the phacoemulsification and the aspiration, aspirated fluids are typically replaced with irrigation of a balanced salt solution, in order to maintain fluid pressure in the anterior chamber. In some cases, if deemed to be necessary, then the capsule is polished. Subsequently, the intraocular lens (IOL) is inserted into the capsule. The IOL is typically foldable and is inserted in a folded configuration, before unfolding inside the capsule. At this stage, the viscoelastic is removed, typically using the suction device that was previously used to aspirate fluids from the capsule. If necessary, the incision(s) is sealed by elevating the pressure inside the bulbus oculi (i.e., the globe of the eye), causing the internal tissue to be pressed against the external tissue of the incision, such as to force closed the incision.

### SUMMARY

In accordance with some applications of the present invention, a robotic unit inserts a diathermic capsulotomy tool into a patient's eye through an incision in the patient's cornea. In accordance with some applications, a computer processor is configured to drive to robotic unit to move the tip of the diathermic capsulotomy tool within the eye, but at the same time to constrain motion of the tool at the location at which the tool is disposed within the incision (this location being referred to herein as the “remote center of motion location of the tool”). For some applications, the computer processor determines the disposition of the tool with respect to the incision by analyzing images of the tool and the patient's eye. For some applications, the computer processor constrains motion of the tool at the remote center of motion location of the tool, such that the edges of the tool are prevented from moving past the edges of the incision.

Typically, a tip of the diathermic capsulotomy tool is moved to form a circle on the patient's anterior lens capsule, while a location of entry of the tool into the patient's eye is maintained within the incision or within an incision zone within the patient's cornea, in accordance with some applications of the present invention. Typically, the tip of the diathermic capsulotomy tool includes a diathermic cutting element that is configured to cut the anterior lens capsule. Typically, the cutting element is an electrode pair via which high-frequency energy (e.g., energy at a frequency of more than 100 kHz, e.g., between 200 kHz and 1 MHZ, or between 300 kHz and 700 kHz) is driven into tissue of the anterior lens capsule to thereby cut the anterior lens capsule. The cutting element is typically surrounded by an insulative material, e.g., a polymeric material, that isolates the cutting element from the exterior of the diathermic capsulotomy tool. As described hereinabove, typically, while the tip of the diathermic capsulotomy tool is moved to form a circle on the patient's anterior lens capsule, the location of entry of the tool into the patient's eye (i.e., the remote center of motion location of the tool) is maintained within the incision or within an incision zone within the patient's cornea. A further constraint is that there is typically a height difference between the incision and the anterior lens capsule. Thus, the robotic unit is configured to move and actuate the diathermic capsulotomy tool, subject to the following constraints:
A) Initially, the tool must be inserted via the incision, and as the tip of the tool is advanced to the anterior lens capsule, the entry of the tool into the patient's eye must remain within the incision (or incision zone). Typically, the tip of the diathermic capsulotomy tool is not straight. Therefore, as the tip of the tool is advanced through the incision, the robotic unit has to advance the tool along a non-linear path in order to maintain the entry of the tool into the patient's eye must within the incision (or incision zone).
B) While activating the cutting element to apply the high-frequency energy to the anterior lens capsule:
   the cutting element is moved in a circle with respect to the anterior lens capsule;
   the end of the tool is held at an angle with respect to the patient's eye, such that the cutting element is disposed on the patient's anterior lens capsule, but the entry of the tool into the patient eye remains within the incision or the incision zone along the y direction (i.e., the “y axis,” which is the direction that is normal to the x direction, which is as defined below); and
   the entry of the tool into the patient eye is constrained such that the entry of the tool into the patient eye remains within the incision or the incision zone along the x direction (i.e., the “x axis,” which is the direction parallel to the incision and at a tangent to the cornea at the incision).

For some applications, the computer processor analyzes images of the patient's eye, determines the location of the patient's visual axis and drives the robotic units to move the cutting element in a circle that is centered on the patient's visual axis. In this regard, it is noted that the patient's visual axis is typically not located directly at the center of the cornea or of the limbus. For some applications, the imaging system includes coaxial light sources. For some such applications, the computer processor determines the location of the patient's visual axis by directing light toward the patient's eye from each of the coaxial light sources. The patient is typically instructed to look at the coaxial light sources, by the computer processor automatically generating an audio instructions, and/or by one of the operators instructing the patient to do so. The computer processor then identifies Purkinje images (i.e., reflections of the light from the structure of the eye) within images of the eye that are acquired by the imaging system. Typically, the computer processor identifies the patient's visual axis as being located at a point about which the Purkinje images are centered. As described hereinabove, the computer processor typically drives the robotic units to move the cutting element in a circle that is centered on the patient's visual axis.

Typically, the robotic unit is configured to move the diathermic capsulotomy tool without violating the above-described constraints on it motion. The robotic unit typically moves the tool through six degrees of freedom (e.g., movement along the x-, y-, and z-axes, as well as pitch, yaw, and roll). Further typically, the computer processor receives images of the diathermic capsulotomy tool and of the patient's eye and analyzes the images, such as to determine (a) the current disposition of the tip with respect to the patient's visual axis, and (b) the current disposition of the remote center of motion location of the tool relative to the incision. Based on the computer processor's analysis of the images, the computer processor drives the diathermic capsulotomy tool to move and/or activates the cutting clement to apply the high-frequency energy to the anterior lens capsule, subject to the constraints described herein.

It is noted that owing to the relatively small dimensions of the eye, and the above-described constraints on the motion of the diathermic capsulotomy tool, the computer processor is typically able to drive the diathermic capsulotomy tool to perform a circular capsulotomy that is centered on the patient's visual axis more precisely than a human surgeon is able to perform these actions. It is further noted that, for some applications, the robotic unit automatically drives the diathermic capsulotomy tool to perform the above described movements (i.e., entry into the eye and then a circular cutting motion while maintaining the remote center of motion location within the incision or the incision zone) automatically, in response to receiving an instruction from an operator, as opposed to the operator controlling the motion of the diathermic capsulotomy tool (in a “master-slave” manner) via a control component.

It is additionally noted that the robotic unit is configured to move and actuate the diathermic capsulotomy tool, subject to the above-described constraints and all while the patient's eye undergoes motion. Typically, the computer processor the computer processor receives images of the diathermic capsulotomy tool and of the patient's eye and analyzes the images, such as to determine motion of the patient's eye undergoes, and to dynamically adjust the disposition and/or the movement of the diathermic capsulotomy tool such as to account for the motion of the patient's eye. For example, the computer processor dynamically adjusts the disposition of the diathermic capsulotomy tool such that the remote center of motion location of the diathermic capsulotomy tool is maintained within the incision, as the patient's eye undergoes motion. Alternatively or additionally, the computer processor dynamically adjusts the circular motion of the cutting element to conform with the motion that the patient's eye undergoes. Further alternatively or additionally, the computer processor dynamically adjusts the circular motion of the cutting element, such that the circular motion of the cutting element remains centered around the patient's visual axis, as the patient's eye undergoes motion.

There is therefore provided, in accordance with some applications of the present invention, apparatus for performing a capsulotomy procedure on an eye of a patient, the apparatus including:
a diathermic capsulotomy tool including a diathermic cutting element disposed at a tip of the diathermic capsulotomy tool;
a robotic unit configured to move the diathermic capsulotomy tool;
an imaging system configured to image the diathermic capsulotomy tool and the patient's eye; and
at least one computer processor that is configured to:
   receive one or more images of the diathermic capsulotomy tool and the patient's eye from the imaging system,
   drive the robotic unit to insert the diathermic capsulotomy tool into the patient's eye via an incision in a cornea of the patient's eye, such that the tip of the diathermic capsulotomy tool is disposed within the patient's eye and a remote center of motion location of the diathermic capsulotomy tool is disposed within the incision, and
   drive the robotic unit to move the cutting element in a circular motion while activating the diathermic cutting element to apply diathermic energy to an anterior lens capsule of the patient's eye, and while maintaining the remote center of motion location of the diathermic capsulotomy tool within the incision.

In some applications, in order to drive the robotic unit to move the cutting element in the circular motion while activating the diathermic cutting element to apply diathermic energy to an anterior lens capsule of the patient's eye, and while maintaining the remote center of motion location of the diathermic capsulotomy tool within the incision, the computer processor is configured to drive the robotic unit to hold the end of the diathermic capsulotomy tool at an angle with respect to the patient's eye, such that the diathermic cutting element is disposed on the anterior lens capsule of the patient's eye, but the entry of the tool into the patient eye remains within the incision.

In some applications, the computer processor is configured to determine motion that the patient's eye undergoes and to dynamically adjust a disposition of the diathermic capsulotomy tool such that the remote center of motion location of the diathermic capsulotomy tool is maintained within the incision.

In some applications, the computer processor is configured to determine motion that the patient's eye undergoes and to dynamically adjust the circular motion of the cutting element to conform with the motion that the patient's eye undergoes.

In some applications, the tip of the diathermic capsulotomy tool is not straight, such that in order to drive the robotic unit to insert the diathermic capsulotomy tool into the patient's eye via the incision, the computer processor is configured to drive the robotic unit to advance the diathermic capsulotomy along a non-linear path.

In some applications, the computer processor is configured to determine a location of a visual axis of the patient, and is configured to move the cutting element in a circular motion by move the cutting element in a circular motion that is centered around the patient's visual axis.

In some applications, the computer processor is configured to determine motion that the subject's eye undergoes and to dynamically adjust the circular motion of the cutting element, such that the circular motion of the cutting element remains centered around the patient's visual axis.

In some applications, the apparatus further includes coaxial light sources disposed on the imaging system, the computer processor is configured to determine the location of the patient's visual axis by directing light toward the patient's eye from the coaxial light sources, identifying Purkinje images within one or more of the images that are acquired by the imaging system, and identifying the patient's visual axis as being located at a point about which the Purkinje images are centered.

There is further provided, in accordance with some applications of the present invention, a method for performing a capsulotomy procedure on an eye of a patient using a diathermic capsulotomy tool that includes a diathermic cutting element disposed at its tip, the method including:
imaging the diathermic capsulotomy tool and the patient's eye using an imaging system; and
using at least one computer processor:
   receiving one or more images of the diathermic capsulotomy tool and the patient's eye from the imaging system;
   driving a robotic unit to insert the diathermic capsulotomy tool into the patient's eye via an incision in a cornea of the patient's eye, such that the tip of the diathermic capsulotomy tool is disposed within the patient's eye and a remote center of motion location of the diathermic capsulotomy tool is disposed within the incision; and
   driving the robotic unit to move the cutting element in a circular motion while activating the diathermic cutting element to apply diathermic energy to an anterior lens capsule of the patient's eye, and while maintaining the remote center of motion location of the diathermic capsulotomy tool within the incision.

In some applications, in order to move the cutting element in a circular motion while activating the diathermic cutting element to apply diathermic energy to an anterior lens capsule of the patient's eye, and while maintaining the remote center of motion location of the diathermic capsulotomy tool within the incision, the method further includes using the computer processor to drive the robotic unit to hold the end of the diathermic capsulotomy tool at an angle with respect to the patient's eye, such that the diathermic cutting element is disposed on the anterior lens capsule of the patient's eye, but the entry of the tool into the patient eye remains within the incision.

In some applications, the method further includes, using the computer processor determining, motion that the patient's eye undergoes and dynamically adjusting a disposition of the diathermic capsulotomy tool such that the remote center of motion location of the diathermic capsulotomy tool is maintained within the incision.

In some applications, the method further includes, using the computer processor determining, motion that the patient's eye undergoes and dynamically adjusting the circular motion of the cutting element to conform with the motion that the patient's eye undergoes.

In some applications, the tip of the diathermic capsulotomy tool is not straight, such that driving a robotic unit to insert the diathermic capsulotomy tool into the patient's eye via an incision in a cornea of the patient's eye includes driving the robotic unit to advance the diathermic capsulotomy along a non-linear path.

In some applications, the method further includes using the computer processor, determining a location of a visual axis of the patient, driving the robotic unit to move the cutting element in the circular motion includes driving the robotic unit to move the cutting element in a circular motion that is centered around the patient's visual axis.

In some applications, the method further includes, using the computer processor determining, motion that the patient's eye undergoes and dynamically adjusting the circular motion of the cutting element, such that the circular motion of the cutting element remains centered around the patient's visual axis.

In some applications, determining the location of the patient's visual axis includes directing light toward the patient's eye from coaxial light sources, identifying Purkinje images within one or more of the images that are acquired by the imaging system, and identifying the patient's visual axis as being located at a point about which the Purkinje images are centered.

The present invention will be more fully understood from the following detailed description of embodiments thereof, taken together with the drawings, in which:

### BRIEF DESCRIPTION OF THE DRAWINGS

FIG. 1 is a schematic illustration of a robotic system that is configured for use in a microsurgical procedure, such as intraocular surgery, in accordance with some applications of the present invention;

FIG. 2 is a schematic illustration of an incision in a patient's cornea, in accordance with some applications of the present invention;

FIG. 3 is a schematic illustration of a robotic unit inserting a diathermic capsulotomy tool into a patient's eye through an incision in the patient's cornea, in accordance with some applications of the present invention; and

FIGS. 4A and 4B are schematic illustrations of the robotic unit moving a diathermic tip of the diathermic capsulotomy tool to form a circle on the patient's anterior lens capsule, while a location of entry of the tool into the patient's eye is maintained within an incision or an incision zone, in accordance with some applications of the present invention.

### DETAILED DESCRIPTION OF EMBODIMENTS

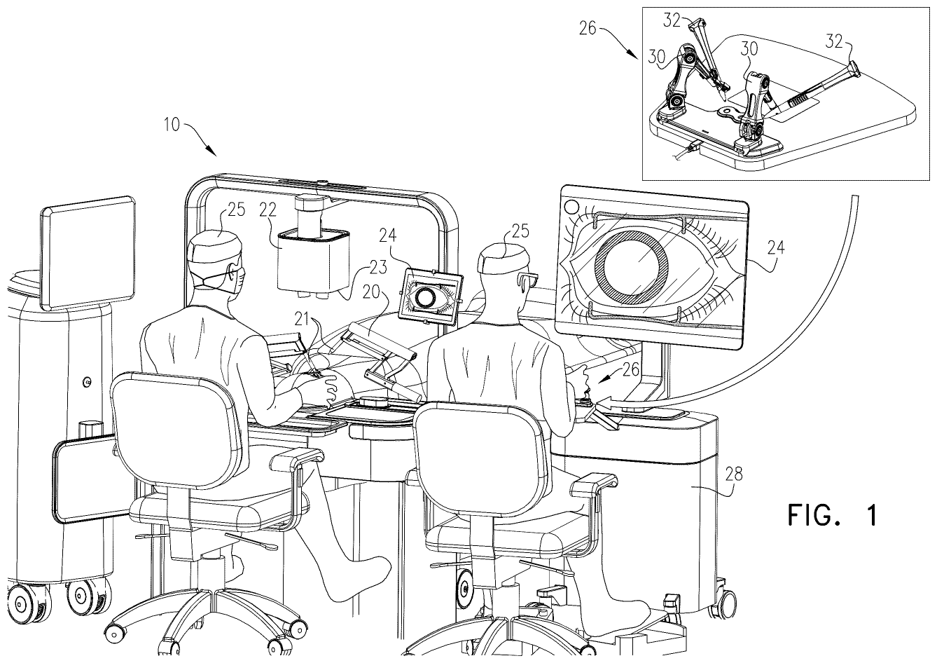

Reference is now made to FIG. 1, which is a schematic illustration of a robotic system 10 that is configured for use in a microsurgical procedure, such as intraocular surgery, in accordance with some applications of the present invention. Typically, when used for intraocular surgery, robotic system 10 includes one or more robotic units 20 (which are configured to hold tools 21), in addition to an imaging system 22, one or more displays 24 and a control component unit 26. Typically, the control component unit includes one or more control components 30 (e.g., a pair of control components 30, as shown in the enlarged portion of FIG. 1), via which one or more operators 25 (e.g., healthcare professionals, such as a physician and/or a nurse) are able to control robotic units 20. Typically, robotic system 10 includes one or more computer processors 28, via which components of the system and operator(s) 25 operatively interact with each other. The scope of the present application includes mounting one or more robotic units in any of a variety of different positions with respect to each other.

Typically, movement of the robotic units (and/or control of other aspects of the robotic system) is at least partially controlled by one or more operators 25 (e.g., healthcare professionals, such as a physician and/or a nurse). For example, the operator may receive images of the patient's eye and the robotic units and/or tools disposed therein, via display 24. Typically, such images are acquired by imaging system 22. For some applications, imaging system 22 is a stereoscopic imaging device and display 24 is a stereoscopic display. Based on the received images, the operator typically performs steps of the procedure. For some applications, the operator provides commands to the robotic units via control component unit 26. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

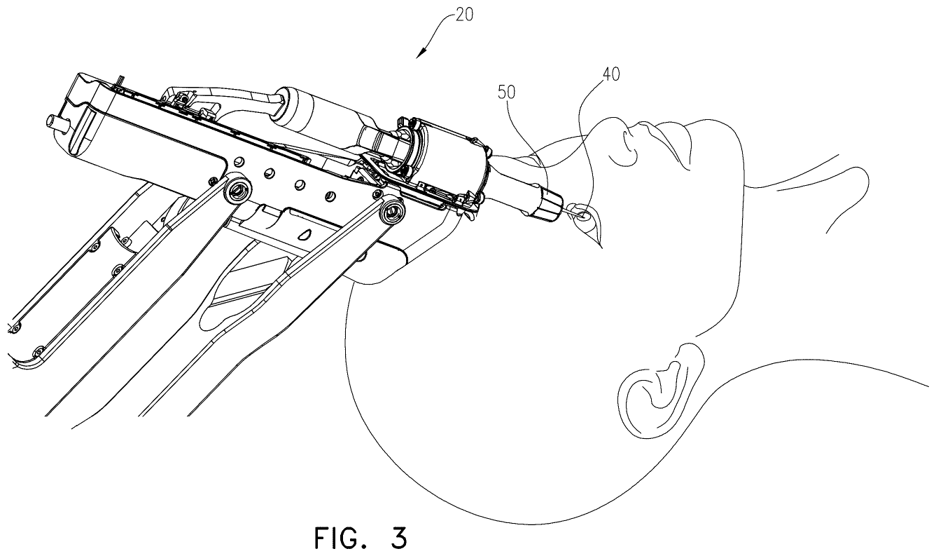

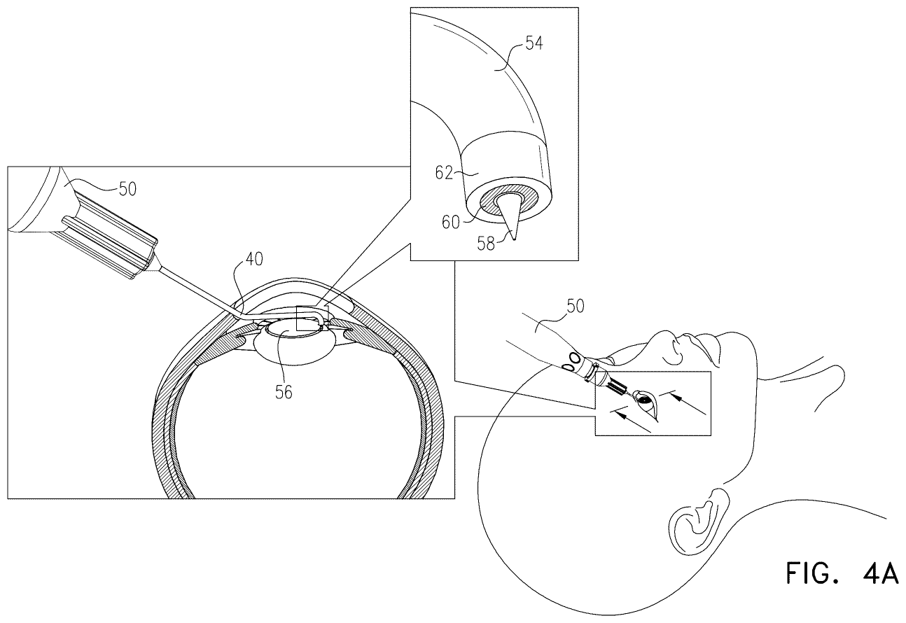

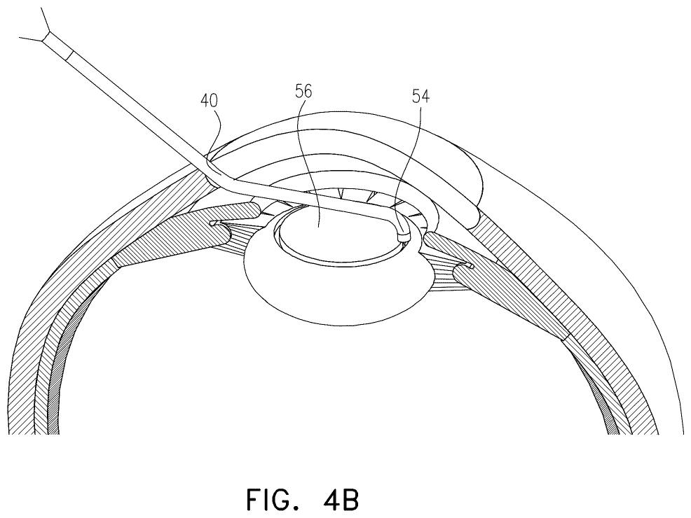

Typically, control component unit 26 includes one or more control components 30 that are configured to correspond to respective robotic units 20 of the robotic system. For example, as shown, the system may include first and second robotic units, and the control component may include first and second control components, as shown. Typically, each of the control components is a control-component arm that includes a plurality of links that are coupled to each other via joints. For some applications, the control-components comprise respective control-component tools 32 therein (in order to replicate the robotic units), as shown in FIG. 1. Typically, the computer processor determines the XYZ location and orientation of the tip of the control-component tool 32, and drives the robotic unit such that the tip of the ophthalmic tool 21 that is being used to perform the procedure tracks the movements of the tip of the control-component tool. For some applications, the robotic units are configured to move the tip of the tool through six degrees of freedom (e.g., movement along the x-, y-, and z-axes, as well as pitch, yaw, and roll). For some applications, in the case of some tools, in response to an input from the use, the computer processor drives the tool to automatically perform a given action with respect to the patient's eye. For example, the computer processor may automatically drive the tip of a diathermic capsulotomy tool 50 (which is an example of ophthalmic tool 21, and which is shown in FIG. 3-FIG. 4B) to cut a circular incision in the anterior lens capsule, in response to a user input, as described in further detail hereinbelow.

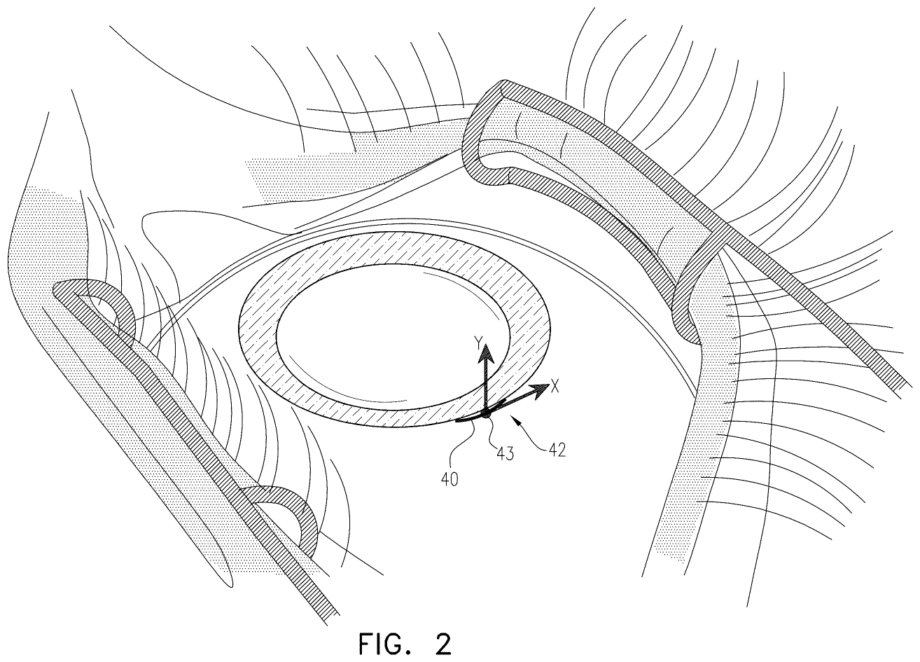

Reference is now made to FIG. 2, which is a schematic illustration of an incision 40 in a patient's cornea 42, in accordance with some applications of the present invention. As described hereinabove in the Background section, typically during a cataract procedure, one or more incisions (and typically two or three incisions) are made in the cornea of the eye. The incision(s) are typically made using a specialized blade, which is called a keratome blade. Typically, the robotic unit is configured to insert tools 21 into the patient's eye such that entry of the tool into the patient's eye is via incision 40, and the tip of the tool is disposed within the patient's eye. Further typically, robotic system 10 is configured to move the tip of the tool within the patient's eye in such a manner that entry of the tool into the patient's eye is constrained to remain within the incision. For some applications, the incision width is equal to the width of the keratome blade. The incision center point 43 is hereby defined as the point on the corneal surface that is centered within the incision widthwise. In FIG. 2, axes have been added, with the x axis parallel to the incision and at a tangent to the cornea at the incision, and the y axis normal to the x axis, and at a tangent to the cornea at the incision. Examples of the present invention will be described hereinbelow with reference to the x and y axes.

In order to perform non-robotic anterior ophthalmic surgery, a surgeon typically makes one or more incisions in the patient's cornea, which is thereafter used as an entry point for various surgical tools. A tool is inserted through an incision, and is manipulated within the eye to achieve the surgical goals. While this manipulation occurs, it is medically preferable that the tool does not forcefully press against the incision edges, lift upwards, or depress downwards exceedingly. Such motions may cause tearing at the incision edges, which widens the incision and can negatively impact the surgical outcome. Ideally, the surgeon will manipulate a tool such that at the entry point of the tool through the incision, the tool is rotated about the center of the incision and not moved laterally, with such motion of the tool at the incision being described herein as maintaining the center of motion. For robotic procedures, such as those described herein, the above-described motion of tool 21 is described as maintaining a remote center of motion, since the tool is typically controlled from a distance (via control component unit 26). In non-robotic procedures, it can be difficult to manually maintain a center of motion, especially when the surgeon needs to focus on the tool tip, which is performing the current surgical action.

Reference is now made to FIG. 3, which is a schematic illustration of robotic unit 20 inserting diathermic capsulotomy tool 50 into a patient's eye through incision 40 in the patient's cornea, in accordance with some applications of the present invention. In accordance with some applications of the present invention, the computer processor is configured to drive to robotic unit to move the tip of diathermic capsulotomy tool 50 within the eye, but at the same time to constrain motion of the tool at the location at which the tool is disposed within the incision (this location being referred to herein as the “remote center of motion location of the tool”). For some applications, the computer processor determines the disposition of the tool with respect to the incision by analyzing images of the tool and the patient's eye. For some applications, the computer processor constrains motion of the tool at the remote center of motion location of the tool, such that the edges of the tool are prevented from moving past the edges of the incision.

Reference is now made to FIGS. 4A and 4B, which are schematic illustrations of the robotic unit moving a tip 54 of the diathermic capsulotomy tool 50 to form a circle on the patient's anterior lens capsule 56, while a location of entry of the tool into the patient's eye is maintained within incision 40 or within an incision zone within the patient's cornea, in accordance with some applications of the present invention. As shown in FIG. 3A, typically, the tip of the diathermic capsulotomy tool includes a diathermic cutting element 58 that is configured to cut the anterior lens capsule. Typically, the cutting element is an electrode pair via which high-frequency energy (e.g., energy at a frequency of more than 100 kHz, e.g., between 200 kHz and 1 MHz, or between 300 kHz and 700 kHz) is driven into tissue of the anterior lens capsule to thereby cut the anterior lens capsule. The cutting element is typically surrounded by an insulative material 60, e.g., a polymeric material, that isolates the cutting element from the exterior 62 of the diathermic capsulotomy tool. As described hereinabove, typically, while the tip 54 of the diathermic capsulotomy tool 50 is moved to form a circle on the patient's anterior lens capsule 56, the location of entry of the tool into the patient's eye (i.e., the remote center of motion location of the tool) is maintained within incision 40 or within an incision zone within the patient's cornea. A further constraint is that there is typically a height difference between the incision and the anterior lens capsule. Thus, the robotic unit is configured to move and actuate the diathermic capsulotomy tool 50, subject to the following constraints:
A) Initially, the tool must be inserted via the incision, and as the tip of the tool is advanced to the anterior lens capsule, the entry of the tool into the patient's eye must remain within the incision (or incision zone). It is noted that, as shown, tip 54 of the diathermic capsulotomy tool 50 is typically not straight. Therefore, as the tip of the tool is advanced through the incision, the robotic unit has to advance the tool along a non-linear path in order to maintain the entry of the tool into the patient's eye must within the incision (or incision zone).
B) While activating the cutting element to apply the high-frequency energy to the anterior lens capsule:
   the cutting element is moved in a circle with respect to the anterior lens capsule;
   the end of the tool is held at an angle with respect to the patient's eye, such that the cutting element is disposed on the patient's anterior lens capsule, but the entry of the tool into the patient eye remains within the incision or the incision zone along the y direction; and
   the entry of the tool into the patient eye is constrained such that the entry of the tool into the patient eye remains within the incision or the incision zone along the x direction.

As described hereinabove, typically, the robotic unit moves cutting element 58 a circle with respect to the anterior lens capsule 56. For some applications, the computer processor analyzes images of the patient's eye, determines the location of the patient's visual axis and drives the robotic units to move the cutting element in a circle that is centered on the patient's visual axis. In this regard, it is noted that the patient's visual axis is typically not located directly at the center of the cornea or of the limbus. For some applications, imaging system 22 (shown in FIG. 1) includes coaxial light sources 23. (In FIG. 1, the light sources themselves are not visible, but they are typically disposed on the surface that faces the patient's eye, as indicated by where reference numeral 23 is pointing toward.) For some such applications, the computer processor determines the location of the patient's visual axis by directing light toward the patient's eye from each of the coaxial light sources. The patient is typically instructed to look at the coaxial light sources, by the computer processor automatically generating an audio instructions, and/or by one of the operators instructing the patient to do so. The computer processor then identifies Purkinje images within images of the eye that are acquired by the imaging system. Typically, the computer processor identifies the patient's visual axis as being located at a point about which the Purkinje images are centered. As described hereinabove, the computer processor typically drives the robotic units to move the cutting element in a circle that is centered on the patient's visual axis.

Typically, the robotic unit is configured to move the diathermic capsulotomy tool 50 without violating the above-described constraints on it motion. As described hereinabove, the robotic unit typically moves the tool through six degrees of freedom (e.g., movement along the x-, y-, and z-axes, as well as pitch, yaw, and roll). Further typically, the computer processor receives images of the diathermic capsulotomy tool 50 and of the patient's eye and analyzes the images, such as to determine (a) the current disposition of the tip with respect to the patient's visual axis, and (b) the current disposition of the remote center of motion location of the tool relative to the incision. Based on the computer processor's analysis of the images, the computer processor drives the diathermic capsulotomy tool 50 to move and/or activates the cutting element to apply the high-frequency energy to the anterior lens capsule, subject to the constraints described herein. It is noted that owing to the relatively small dimensions of the eye, and the above-described constraints on the motion of the diathermic capsulotomy tool 50, the computer processor is typically able to drive the diathermic capsulotomy tool 50 to perform a circular capsulotomy that is centered on the patient's visual axis more precisely than a human surgeon is able to perform these actions. It is further noted that, for some applications, the robotic unit automatically drives the diathermic capsulotomy tool 50 to perform the above described movements (i.e., entry into the eye and then a circular cutting motion while maintaining the remote center of motion location within the incision or the incision zone) automatically, in response to receiving an instruction from an operator, as opposed to the operator controlling the motion of the diathermic capsulotomy tool 50 (in a “master-slave” manner) via the control component 30.

It is additionally noted that the robotic unit is configured to move and actuate the diathermic capsulotomy tool 50, subject to the above-described constraints and all while the patient's eye undergoes motion. Typically, the computer processor the computer processor receives images of the diathermic capsulotomy tool 50 and of the patient's eye and analyzes the images, such as to determine motion of the patient's eye undergoes, and to dynamically adjust the disposition and/or the movement of the diathermic capsulotomy tool 50 such as to account for the motion of the patient's eye. For example, the computer processor dynamically adjusts the disposition of the diathermic capsulotomy tool such that the remote center of motion location of the diathermic capsulotomy tool is maintained within the incision, as the patient's eye undergoes motion. Alternatively or additionally, the computer processor dynamically adjusts the circular motion of the cutting element to conform with the motion that the patient's eye undergoes. Further alternatively or additionally, the computer processor dynamically adjusts the circular motion of the cutting element, such that the circular motion of the cutting element remains centered around the patient's visual axis, as the patient's eye undergoes motion.

Although some applications of the present invention are described with reference to cataract surgery, the scope of the present application includes applying the apparatus and methods described herein to other medical procedures, mutatis mutandis. In particular, the apparatus and methods described herein to other medical procedures may be applied to other microsurgical procedures, such as general surgery, orthopedic surgery, gynecological surgery, otolaryngology, neurosurgery, oral and maxillofacial surgery, plastic surgery, podiatric surgery, vascular surgery, and/or pediatric surgery that is performed using microsurgical techniques. For some such applications, the imaging system includes one or more microscopic imaging units.

It is noted that the scope of the present application includes applying the apparatus and methods described herein to intraocular procedures, other than cataract surgery, mutatis mutandis. Such procedures may include collagen crosslinking, endothelial keratoplasty (e.g., DSEK, DMEK, and/or PDEK), DSO (descemet stripping without transplantation), laser assisted keratoplasty, keratoplasty, LASIK/PRK, SMILE, pterygium, ocular surface cancer treatment, secondary IOL placement (sutured, transconjunctival, etc.), iris repair, IOL reposition, IOL exchange, superficial keratectomy, Minimally Invasive Glaucoma Surgery (MIGS), limbal stem cell transplantation, astigmatic keratotomy, Limbal Relaxing Incisions (LRI), amniotic membrane transplantation (AMT), glaucoma surgery (e.g., trabs, tubes, minimally invasive glaucoma surgery), automated lamellar keratoplasty (ALK), anterior vitrectomy, and/or pars plana anterior vitrectomy.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described with reference to the Figures, computer processor 28 typically acts as a special purpose robotic-system computer processor. Typically, the operations described herein that are performed by computer processor 28 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for performing a capsulotomy procedure on an eye of a patient, the apparatus comprising:
a diathermic capsulotomy tool comprising a diathermic cutting element disposed at a tip of the diathermic capsulotomy tool;
a robotic unit configured to move the diathermic capsulotomy tool;
an imaging system configured to image the diathermic capsulotomy tool and the patient's eye; and
at least one computer processor that is configured to:
receive one or more images of the diathermic capsulotomy tool and the patient's eye from the imaging system,
drive the robotic unit to insert the diathermic capsulotomy tool into the patient's eye via an incision in a cornea of the patient's eye, such that the tip of the diathermic capsulotomy tool is disposed within the patient's eye and a remote center of motion location of the diathermic capsulotomy tool is disposed within the incision, and
drive the robotic unit to move the cutting element in a circular motion while activating the diathermic cutting element to apply diathermic energy to an anterior lens capsule of the patient's eye, and while maintaining the remote center of motion location of the diathermic capsulotomy tool within the incision.

2. The apparatus according to claim 1, wherein in order to drive the robotic unit to move the cutting element in the circular motion while activating the diathermic cutting element to apply diathermic energy to an anterior lens capsule of the patient's eye, and while maintaining the remote center of motion location of the diathermic capsulotomy tool within the incision, the computer processor is configured to drive the robotic unit to hold the end of the diathermic capsulotomy tool at an angle with respect to the patient's eye, such that the diathermic cutting element is disposed on the anterior lens capsule of the patient's eye, but the entry of the tool into the patient eye remains within the incision.

3. The apparatus according to claim 1, wherein the computer processor is configured to determine motion that the patient's eye undergoes and to dynamically adjust a disposition of the diathermic capsulotomy tool such that the remote center of motion location of the diathermic capsulotomy tool is maintained within the incision.

4. The apparatus according to claim 1, wherein the computer processor is configured to determine motion that the patient's eye undergoes and to dynamically adjust the circular motion of the cutting element to conform with the motion that the patient's eye undergoes.

5. The apparatus according to claim 1, wherein the tip of the diathermic capsulotomy tool is not straight, such that in order to drive the robotic unit to insert the diathermic capsulotomy tool into the patient's eye via the incision, the computer processor is configured to drive the robotic unit to advance the diathermic capsulotomy along a non-linear path.

6. The apparatus according to claim 1, wherein the computer processor is configured to determine a location of a visual axis of the patient, and is configured to move the cutting element in a circular motion by move the cutting element in a circular motion that is centered around the patient's visual axis.

7. The apparatus according to claim 6, wherein the computer processor is configured to determine motion that the subject's eye undergoes and to dynamically adjust the circular motion of the cutting element, such that the circular motion of the cutting element remains centered around the patient's visual axis.

8. The apparatus according to claim 6, further comprising coaxial light sources disposed on the imaging system, wherein the computer processor is configured to determine the location of the patient's visual axis by directing light toward the patient's eye from the coaxial light sources, identifying Purkinje images within one or more of the images that are acquired by the imaging system, and identifying the patient's visual axis as being located at a point about which the Purkinje images are centered.

9. A method for performing a capsulotomy procedure on an eye of a patient using a diathermic capsulotomy tool that includes a diathermic cutting element disposed at its tip, the method comprising:
imaging the diathermic capsulotomy tool and the patient's eye using an imaging system; and
using at least one computer processor:
receiving one or more images of the diathermic capsulotomy tool and the patient's eye from the imaging system;
driving a robotic unit to insert the diathermic capsulotomy tool into the patient's eye via an incision in a cornea of the patient's eye, such that the tip of the diathermic capsulotomy tool is disposed within the patient's eye and a remote center of motion location of the diathermic capsulotomy tool is disposed within the incision; and
driving the robotic unit to move the cutting element in a circular motion while activating the diathermic cutting clement to apply diathermic energy to an anterior lens capsule of the patient's eye, and while maintaining the remote center of motion location of the diathermic capsulotomy tool within the incision.

10. The method according to claim 9, wherein in order to move the cutting element in a circular motion while activating the diathermic cutting element to apply diathermic energy to an anterior lens capsule of the patient's eye, and while maintaining the remote center of motion location of the diathermic capsulotomy tool within the incision, the method further comprises using the computer processor to drive the robotic unit to hold the end of the diathermic capsulotomy tool at an angle with respect to the patient's eye, such that the diathermic cutting element is disposed on the anterior lens capsule of the patient's eye, but the entry of the tool into the patient eye remains within the incision.

11. The method according to claim 9, further comprising, using the computer processor determining, motion that the patient's eye undergoes and dynamically adjusting a disposition of the diathermic capsulotomy tool such that the remote center of motion location of the diathermic capsulotomy tool is maintained within the incision.

12. The method according to claim 9, further comprising, using the computer processor determining, motion that the patient's eye undergoes and dynamically adjusting the circular motion of the cutting element to conform with the motion that the patient's eye undergoes.

13. The method according to claim 9, wherein the tip of the diathermic capsulotomy tool is not straight, such that driving a robotic unit to insert the diathermic capsulotomy tool into the patient's eye via an incision in a cornea of the patient's eye comprises driving the robotic unit to advance the diathermic capsulotomy along a non-linear path.

14. The method according to claim 9, further comprising using the computer processor, determining a location of a visual axis of the patient, wherein driving the robotic unit to move the cutting element in the circular motion comprises driving the robotic unit to move the cutting element in a circular motion that is centered around the patient's visual axis.

15. The method according to claim 14, further comprising, using the computer processor determining, motion that the patient's eye undergoes and dynamically adjusting the circular motion of the cutting element, such that the circular motion of the cutting element remains centered around the patient's visual axis.

16. The method according to claim 14, wherein determining the location of the patient's visual axis comprises directing light toward the patient's eye from coaxial light sources, identifying Purkinje images within one or more of the images that are acquired by the imaging system, and identifying the patient's visual axis as being located at a point about which the Purkinje images are centered.
